(19) United States
(12) Patent Application Publication
(10) Pub. No.: US 2002/0179086 A1
(43) Pub. Date: Dec. 5, 2002

(54) Method of medicine administration for respiratory tract

(76) Inventor: Shih-Ho Chang, ChangHua (TW)

Correspondence Address: DOUGHERTY & TROXELL, 5205 LEESBURG PIKE, SUITE 1404, FALLS CHURCH, VA, 22041, US

(21) Appl. No.: 09/867,410

(22) Filed: May 31, 2001

(51) Int. Cl. B05D 7/14; A61M 15/00

(52) U.S. Cl. 128/203.15

(57) ABSTRACT

The present invention relates to a method of medicine administration for respiratory tract and an apparatus for continuous medicine administration for respiratory tract. The present invention mainly utilize a respiratory tract medicine-administrated apparatus, which includes a medicine-storing chamber, which may be attached with an opening for filling in medicine, a medicine-administrated mechanism, which is connected to the medicine-storing chamber, and a medicine-exiting port, which is connected to the medicine-administrated mechanism. The medicine-administrated mechanism includes a pusher, an air intake, and a medicine-mixing chamber. In the most front end of the medicine-exiting port, there is an arrangement of nose-inserting portion of medicine-inhaling hole with opening shape. The interior of medicine-storing chamber may be filled with powder-like medicine, and a pusher with handgrip is inserted into one side portion of the medicine-storing chamber. The medicine-mixing chamber is connected to the pusher's trough. On the upper portion of the medicine-mixing chamber, there is an arrangement of air intake for introducing air. Wherein, the medicine storing chamber is already added with powder-like medicine, and the medicine is also dropped into the medicine-retrieving portion of the pusher, after the pusher is pushed back, the medicine will drop into the medicine-mixing chamber for proceeding mixing. By inserting the nose-inserting portion into the nostril and inhaled, the medicine is mixed with air, inhaled into the nostril through the medicine-inhaling hole, entering into human body, and adhered to the mucous membrane of the respiratory tract for effectively developing the effect of the medicine. With the respiratory tract medicine-administrated method of the present invention, the medicine will be directly inhaled into the respiratory tract and adhered onto the mucous membrane to optimize the treatment for the diseases of respiratory tract.

### BACKGROUND OF THE INVENTION

1. Field of the Invention

The present invention relates to a method of medicine administration for respiratory tract and an apparatus for continuous medicine administration for respiratory tract, more particularly, the present invention relates to an apparatus for medicine administration for respiratory tract, utilizing the powder-like antibiotics or bacteriophage, of which characteristic is easily to be adhered onto the mucous membrane of the respiratory tract, and which are inhaled directly into the respiratory tract through nasal cavity or mouth with the apparatus of the present invention for medicine administration for respiratory tract to reach the excellent effects.

2. Description of the Prior Art

The common infectious disease of respiratory tract is indeed a great harassment for most people. Often the disease such as influenza etc. has brought lots of inconvenience to the individuals. The lightly infected patients will recover within few days, however, for most people, they must visit doctors for treatment and take several days to recover completely, but it is possible to be recurrent. Furthermore, it will induce more serious diseases of bronchus or lung etc.

To improve the described above, the present invention provides a new and effective method. A concept must be clarified before the present invention is described: traditionally, the viewpoints for the infectious diseases in respiratory tract are very incomplete and misunderstanding. Actually, the mechanism of viral infection sometimes is undivided with those of bacteria. Therefore, in the treatment of the relative diseases, if we insist that the virus has nothing to do with the bacteria, there will be serious negligence in treatment procedures and causing poor effect.

However, the influenza virus attacks the mucous membrane and induces the immune system of the human body to fight exhaustedly against the invasion of the bacteria which is at the most frontline of the immune reaction. On the contrary, the virus, hiding behind the mucous cells, withdraws to the second line and takes this chance to replication excessively. Therefore, the potency of the well-known oral medicine such like antibiotics is poor, and it usually takes over several days after taking oral medicine to show apparent effect.

According to above-mentioned description, the traditional methods of medicine-administrated remain something wrong and need to be improved. Based on to lots of experiments, the present applicant gives up the traditional medicine-administrated methods and uniquely adopts a novel respiratory tract medicine-administrated apparatus. The result is very satisfactory and the apparatus is very useful; that is, the antibiotics not only can fight against the bacteria but also suppress the infection of influenza viruses. So it is very effective to cure the influenza in a short time. Therefore, the present invention provides a method of the medicine administration for respiratory tract and a apparatus for continuous medicine administration for respiratory tract, and it is very potent to treat those respiratory tract diseases such like influenza etc.

### SUMMARY OF INVENTION

It is therefore an objective of the present invention is to provide a method of the medicine administration for respiratory tract and a apparatus for continuous medicine administration for respiratory tract, utilizing the powder-like antibiotics or bacteriophage, of which characteristic is easily to be adhered onto the mucous membrane of the respiratory tract, and which are inhaled directly into the respiratory tract through nasal cavity or mouth with the apparatus of the present invention for medicine administration for respiratory tract to reach the excellent effects.

Another objective of the present invention is to provide a method of the medicine administration for respiratory tract and a apparatus for continuous medicine administration for respiratory tract, which can make the medicine be absorbed completely by the respiratory tract of human body and may fight against the bacteria in respiratory tract to reach the excellent function of suppressing infectious diseases.

To reach above-mentioned objectives, the present invention is a method of the medicine administration for respiratory tract and a apparatus for continuous medicine administration for respiratory tract. In the preferable embodiment, which mainly says the application of a respiratory tract medicine-administrated apparatus, which includes a medicine-storing chamber, which may be attached with an opening for filling in medicine, a medicine-administrated mechanism, which is connected to the medicine-storing chamber, and a medicine-exiting port, which is connected to the medicine-administrated mechanism. The medicine-administrated mechanism includes a pusher, an air intake, and a medicine-mixing chamber. In the most front end of the medicine-exiting port, there is an arrangement of nose-inserting portion of medicine-inhaling hole with opening shape. The interior of medicine-storing chamber may be filled with powder-like medicine, and a pusher with handgrip is inserted into one side portion of the medicine-storing chamber. The medicine-mixing chamber is connected to the pusher's trough. On the upper portion of the medicine-mixing chamber, there is an arrangement of air intake for introducing air. Wherein, the medicine storing chamber is already added with powder-like medicine, and the medicine is also dropped into the medicine-retrieving portion of the pusher, after the pusher is pushed back, the medicine will drop into the medicine-mixing chamber for proceeding mixing. By inserting the nose-inserting portion into the nostril and inhaled, the medicine is mixed with air, inhaled into the nostril through the medicine-inhaling hole, entering into human body, and adhered to the mucous membrane of the respiratory tract for effectively developing the effect of the medicine. With the respiratory tract medicine-administrated method of the present invention, the medicine will be directly inhaled into the nostril through the medicine-inhaling holes, entering into the human body and adhered to the mucous membrane of respiratory tract to reach the curing effect for suppressing and destroying the diseases of respiratory tract.

### BRIEF DESCRIPTION OF THE DRAWINGS

FIG. 1 is the illustration for the method of the respiratory tract medicine-administrated and the respiratory tract medicine-administrated apparatus for continuous usage of the first embodiment of the present invention, wherein the powder-like medicine is filling into the medicine-retrieving portion.

FIG. 2 is the illustration for the method of respiratory tract medicine-administrated and the respiratory tract medicine-administrated apparatus for continuous usage of the first embodiment of the present invention, wherein the sending pusher is filling powder-like medicine into the medicine-mixing chamber.

FIG. 3 is the three-dimensional illustration for the apparatus of respiratory tract medicine-administrated of the first embodiment of the present invention.

FIG. 4 is the illustration for the method of respiratory tract medicine-administrated and the respiratory tract medicine-administrated apparatus for continuous usage of the second embodiment of the present invention, wherein the powder-like medicine is filling into the medicine-retrieving portion.

FIG. 5 is the illustration for the method of respiratory tract medicine-administrated and the respiratory tract medicine-administrated apparatus for continuous usage of the second embodiment of the present invention, wherein the sending pusher is filling powder-like medicine into the medicine-mixing chamber.

FIG. 6 is the three-dimensional illustration for the apparatus of respiratory tract medicine-administrated of the second embodiment of the present invention.

FIG. 7 is the three-dimensional illustration for the apparatus of respiratory tract medicine-administrated of the second embodiment of the present invention.

### DETAILED DESCRIPTION OF A PREFERRED EMBODIMENT

For your esteemed review committee to further understand and recognize the objective, characteristic and function of the present invention, a detailed description matching with drawings are presented as following. Of course, the present invention may be implemented with various methods, and not limited within the said content of this application. The disclosure of this application is very complete, and can completely express the intentionally disclosed spirit of the present invention.

For a method of respiratory tract medicine-administrated and an apparatus of respiratory tract medicine-administrated for continuous usage of the present invention, a detailed description is made. The present invention provides an excellent efficacy that can make medicine be completely absorbed by the respiratory tract of human body to suppress the bacteria in respiratory tract and further suppress the infected virus. But why suppressing or destroying the bacteria in the respiratory tract may suppress the action of the influenza virus. This viewpoint is seemingly different to current knowledge, but the experiment result is still positive. In studying the causes, the applicant considers that there are two major factors as following:

1. From the theory of immunology, the influenza virus firstly adheres to the mucous membrane on respiratory tract of human body and reproduces after invading into the mucous membrane. Simultaneously, it attacks the mucous membrane and produces wounds. Next and shortly, the bacteria invade the wounds and induce the inflammatory response. In the meantime, the blood vessel nearby the wounds will be expanded and the permeability between the tissues is enhanced. Much of the body fluids flow into the wounding area and various white blood cells also move to the same place. Wherein, phagocytes will permeate into tissues through the blood vessel walls and phagocytize the invading bacteria. Among these, C-reactive protein, one of the acute-phase proteins secreted from liver, probably combined with the C-polysaccharide, and then activates the complement system of our bodies. The complement may not only dissolve the cell wall but can promote the phagocytosis of the bacteria. While macrophage proceeds with phagocytosis, the bacteria will be decomposed into many small fragments as a result of acid circumstance. Some part of fragments (i.e. peptides) will be combined with Class II MHC (Major Histocompatibility Complex) into a complex. At this time, the macrophage will secrete a kind of protein called Interleukin (IL-I) to activate the helper T cell (T_{H}). This IL-I will also influence the hypothalamus's thermotaxis center to cause the fever phenomenon of human body. The antigen receptor (TCR) on T_{H} cell is combined with the said antigen-Class II MHC complex. T_{H} also secretes IL-2, IL-4, IL-5, IL-6 and interferon gamma (IFN-γ) to stimulate and activate B cell. Plasma cell is formed to generate antibody. However, since the generated antibody aims to the bacteria, so it fails to fight against the influenza virus. In another word, said influenza virus produces wounds in the mucous membrane to make the human immune system exhaust all its force to fight against the invasion of the bacteria and make the bacteria be placed in the most front line of immune reaction. By doing so, contrarily, the virus, hiding behind the mucous membrane cells, withdraws to the second line and takes this chance to reproduce itself unscrupulously and excessively.

2. Influenza virus is a virus with envelope, so it is very sensitive to the surrounding factors. In order to survive in human body, like HIV that may seize protein (i.e. class I MHC and class II MHC) on the cell surface away human body, except for thickening envelope to avoid being dissolved by MAC (membrane-attack complex) of the human complement system, it also has the usefulness of disguise to make the phagocytes and T cells can not recognize it immediately and phagocytize it, thus let the virus do have sufficient time to invade into cell and hide inside the tissue. Therefore, the virus with envelope such like influenza virus or herpes simplex virus will adhere to the cell mucous membrane or to the skin to produce wounds by destroying the human tissues and the human phagocytes generate plenty class II MHC molecules due to phagocytizing plenty bacteria. When there are sufficient classes II MHC on the outer surface of infected cells, the virus then precedes the budding reproduction to secure every new virus possessing sufficient class II MHC and to protect virus being able to live in human body. Therefore, if in the moment of virus creating wounds, sterilization is proceeding immediately to prevent the phagocyte in wounds from generating plenty class II MHC due to phagocytizing bacteria, then influenza virus can not reproduce class II MHC and either can reproduce itself in mass. The infected cells are finally dissolved and killed by Cytotoxic T Lymphocyte (CTL) to further make influenza virus been controlled and recover soon.

In fact, above-mentioned theoretical model of influenza virus also could explain to the pathogenic mechanism of AIDS. For instance, we know that small amount of HIV could invade and cause viral infection via the skin wounds of human body. But there are few medical staffs to be infected AIDS via syringe needle which with more infection of HIV. It is because that the stab wounds are new, the MHC molecule has not vastly increased by phagocytosis. Therefore, there will be insufficient amount of MHC molecule for HIV to camouflage. As a result, HIV can be completely vanished.

From above-mentioned analysis, it is acknowledged that the activation of influenza virus has close relation with the bacteria theoretically.

### First Embodiment

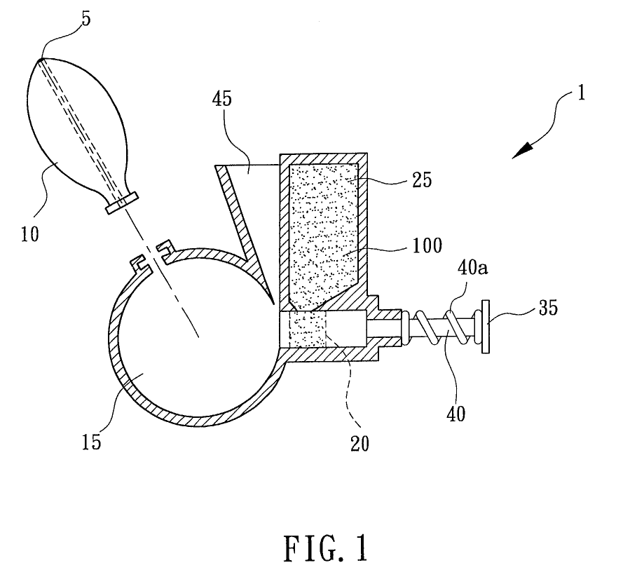

For above-mentioned reflection and verified experiment, a new medicine-administrated apparatus is designed. Please refer to FIG. 1, which shows the illustration for the respiratory tract medicine-administrated apparatus of the first embodiment of the present invention. The said medicine-administrated apparatus 1 in respiratory tract may apply a transparent resin as supporting material and mainly includes a medicine-storing chamber 25, a medicine-administrated mechanism, and a medicine-exiting port. The medicine-administrated mechanism includes a pusher 40, an air intake 45, a medicine-mixing chamber 15; the medicine-exiting port includes a nose-inserting portion 10. The interior of medicine-storing chamber 25 may contain any powder-like medicine 100. The front portion of the pusher 40 has a handgrip 35, on which a design of spring 40a may be attached. Wherein a screw thread 35a may be on the handgrip 35. As making the pusher 40 added with the spring 40a, the screw thread 35a is further locked onto it to make it be locked. Or, the handgrip 35 is fixed with the pusher 40 by gluing; and a medicine-retrieving portion 20 with a hollow opening may be arranged in the rear portion of the pusher 40. The front portion of the medicine-mixing chamber 15 may be connected and fixed with a nose-inserting portion 10 with a shape of elliptical ball or round ball, while the most front end of the nose-inserting portion 10 is arranged with a medicine-inhaling hole 5 with an opening shape. And, an air intake 45 is arranged in the side portion of the medicine-mixing chamber 15. One end of the air intake 45 is an opening. And, the design of the bottom portion of the air intake 45 is a contracting port that makes the air intake 45 may introduce air. When the air enters into the port, it will create a phenomenon of air compression in the bottom portion. FIG. 1 shows the mechanism that the said respiratory tract medicine-administrated apparatus 1 fills the powder-like medicine 100 into the medicine-retrieving portion 20. Wherein, when the powder-like medicine 100 of the medicine-storing chamber 25 is dropped into the medicine-retrieving portion 20, the volume of the medicine-retrieving portion 20 may be designed as a unit quantity of medicine for the measuring standard quantity of filling medicine at each time.

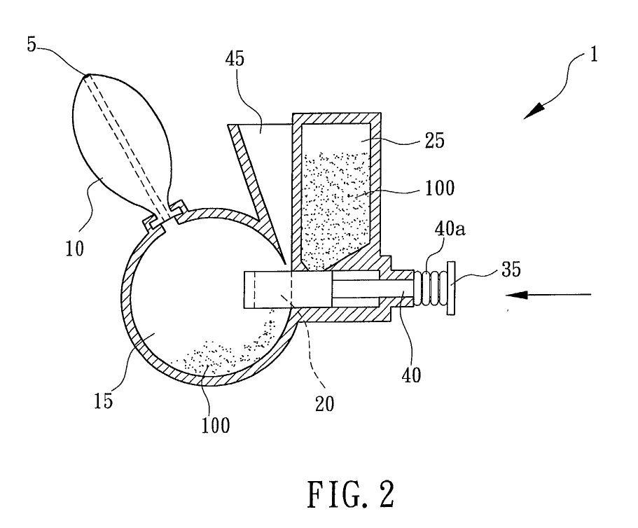

In following, please refer to FIG. 2, which is a mechanism for respiratory tract medicine-administrated apparatus 1 to push forward pusher for filling powder-like medicine 100 into the medicine-mixing chamber 15. Wherein, the pusher 40 is pushed forward with the handgrip 35, at this time, the spring 40a, attached onto the pusher 40, shows a state of compressing, therefore, the powder-like medicine 100, placed in the medicine-retrieving portion, drops into the medicine-mixing chamber 15. When the handgrip 35 is released backward, the recovering force of spring 40a that is attached to the pusher 40 makes the pusher 40 recovers to the original position state of FIG. 1.

In the following, which is the application method for the respiratory tract medicine-administrated apparatus 1. The nose-inserting portion 10 of the respiratory tract medicine-administrated apparatus 1 is inserted into the nostril and the air is inhaled into the medicine-mixing chamber 15 through the air intake 45. The compressed air will create a phenomenon of gas circling to stir up the powder-like medicine 100 and mix it with the air. The stirring and mixing powder-like medicine 100 is inhaled into the nostril through the medicine-inhaling hole 5, enters into human body and adheres to the mucous membrane of respiratory tract. Thus, the method for the respiratory tract medicine-administrated apparatus 1 applied to the medicine that is absorbed by the respiratory tract of human body is then completed.

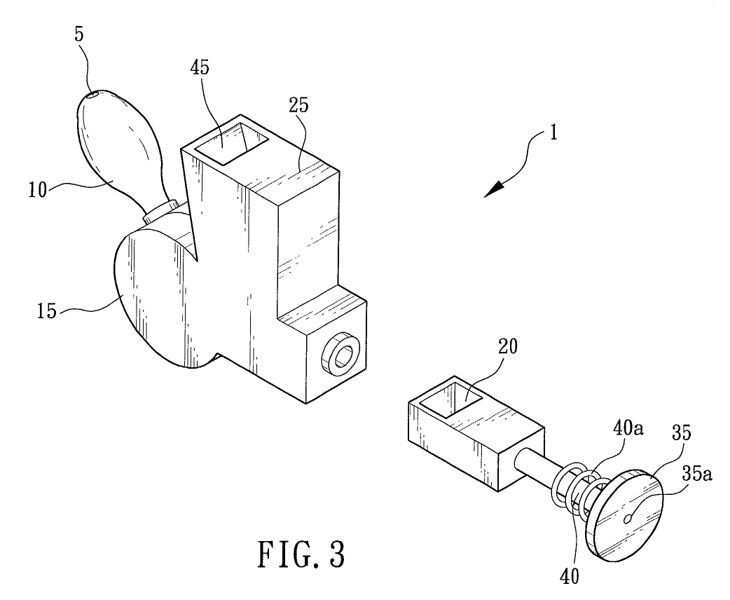

FIG. 3 shows a three-dimensional illustration for the respiratory tract medicine-administrated apparatus 1. Wherein, the pusher 40 has a handgrip 35 for pushing and pulling the pusher 40, on which a spring 40a is designed. When the pusher 40 is being pushed forward, the spring 40a, attached onto the pusher 40, shows a state of compression, and the filling medicine quantity is just the volume of the medicine-retrieving portion 20 (i.e. a unit medicine quantity), therefore the powder-like medicine 100 drops into the medicine-mixing chamber 15 for proceeding air mixing process. When the handgrip 35 is released, the recovering force of the spring 40a that is attached onto the pusher 40 makes the pusher 40 recover to its original position state. And, since the diameter of the exiting port of pusher 40 is smaller than the rear portion of the pusher 40, so the pusher will not go so far as dropping off to facilitate the motion of pushing and pulling to be further steady and smooth. When the pusher 40 pushes the powder-like medicine 100 into the medicine-mixing chamber 15 and the nose-inserting portion 10 is inserted into nose, and then inhaling, only the air may enter into the medicine-mixing chamber 15 through the air intake 45.

### Second Embodiment

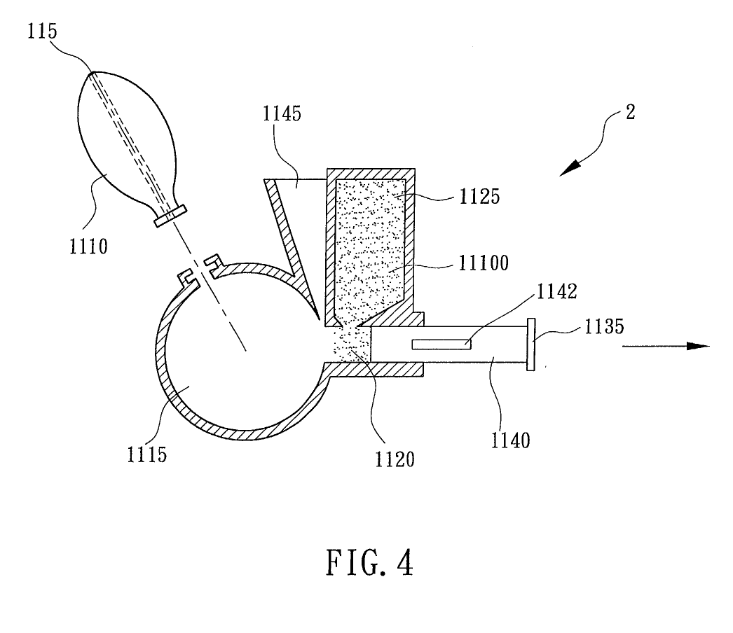

Please refer to FIG. 4, which shows the illustration for the respiratory tract medicine-administrated apparatus of the second embodiment of the present invention. Similarly, the said breathing medicine-administrated apparatus 2 may apply a transparent resin as making material and mainly includes a medicine-storing chamber 1125, a medicine-administrated mechanism, and a medicine-exiting port. The medicine-administrated mechanism includes a pusher 1140, an air intake 1145, a medicine-mixing chamber 1115; the medicine-exiting port includes a nose-inserting portion 1110. The interior of medicine-storing chamber 25 may contain any powder-like medicine 11100. The front portion of the pusher 1140 has a handgrip 1135. The front portion of the medicine-mixing chamber 1115 then may be connected and fixed with a nose-inserting portion 1110 with a shape of elliptical ball or round ball, while the most front end of the nose-inserting portion 1110 is arranged with a medicine-inhaling hole 115 with an opening shape. And, an air intake 1145 is arranged in the side portion of the medicine-mixing chamber 15. One end of the air intake 1145 is an opening. And, the design of the bottom portion of the air intake 1145 is a contracting port that makes the air intake 1145 may introduce air. When the air enters into the port, it will create a phenomenon of air compression in the bottom portion. FIG. 1 shows the mechanism that the said respiratory tract medicine-administrated apparatus 2 fills the powder-like medicine 11100 into the pusher trough 1120. Wherein, the powder-like medicine 100 of the medicine-storing chamber 25 is dropped into the pusher trough 1120.

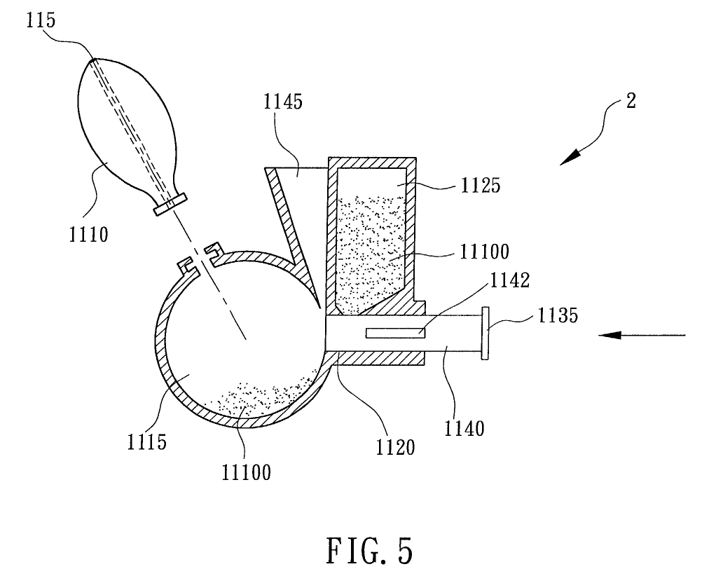

In following, please refer to FIG. 5, which is a mechanism for respiratory tract medicine-administrated apparatus 2 to push forward pusher for filling powder-like medicine 11100 into the medicine-mixing chamber 1115. Wherein, the pusher 40 is sent into with the handgrip 35, therefore the powder-like medicine 11100 drops into the medicine-mixing chamber 1115 for proceeding the necessary air mixing.

In the following, which is the application method for the respiratory tract medicine-administrated apparatus 2. The nose-inserting portion 1110 of the respiratory tract medicine-administrated apparatus 2 is inserted into the nostril and the air is inhaled into the medicine-mixing chamber 1115 through the air intake 1145. The compressed air will create a phenomenon of gas circling to stir up the powder-like medicine 11100 and mix it with the air. The stirring and mixing powder-like medicine 11100 is inhaled into the nostril through the medicine-inhaling hole 115, enters into human body and adheres to the mucous membrane of respiratory tract. Thus, the method for the respiratory tract medicine-administrated apparatus 2 applied to the medicine that is absorbed by the respiratory tract of human body is then completed.

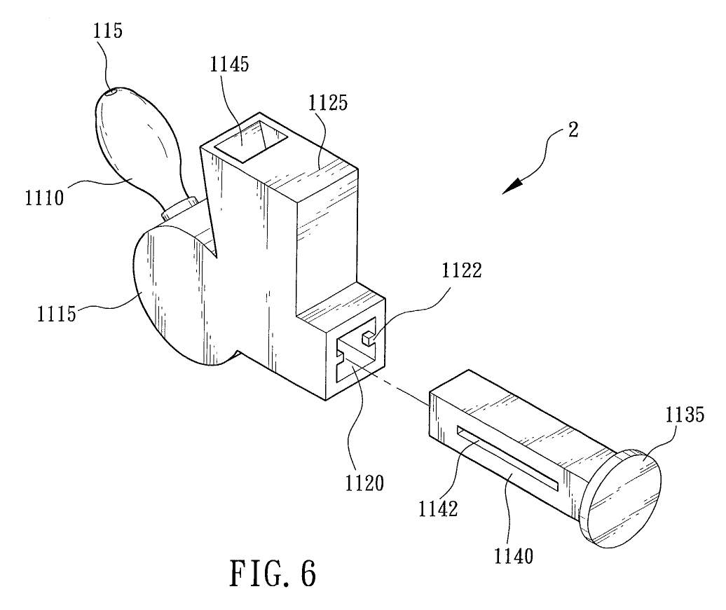

FIG. 6 shows a three-dimensional illustration for the respiratory tract medicine-administrated apparatus 2. Wherein, the pusher 1140 has a handgrip 1135 for pushing and pulling the pusher 1140. And, two sides of the pusher 1140 each has pusher groove 1142, of which length is one half (1/2) to two third (2/3) of the total length of the pusher 1140. And, two sides of the pusher trough 1120 each has a tenon 1122 that may be inserted into the pusher groove 1142 of the pusher 1140 to prevent the pulling out pusher 1140 from dropping off to facilitate the motion of pushing and pulling to be further steady and smooth. When the pusher 1140 pushes the powder-like medicine 11100 into the medicine-mixing chamber 1115 and nose-inserting portion 1110 is inserted into nose, then inhaling, only the air may enters into the medicine-mixing chamber 1115 through the air intake 1145.

### Third Embodiment

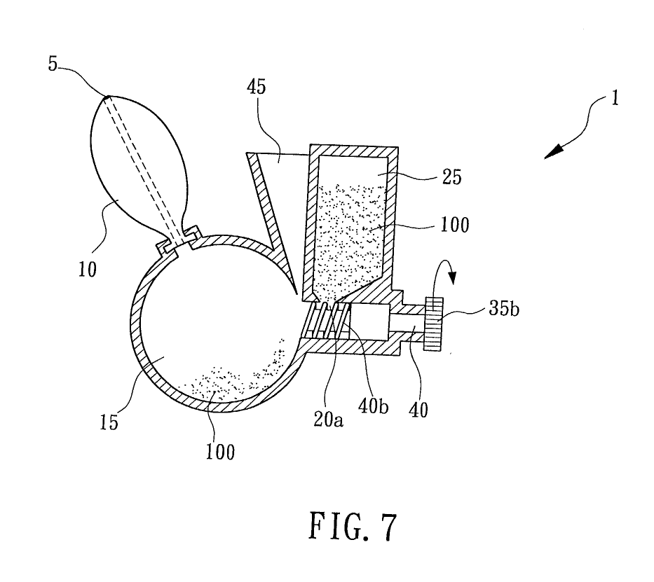

In addition, the present invention can also be implemented with a third embodiment. Please refer to FIG. 7, similarly, the respiratory tract medicine-administrated apparatus 1 also mainly includes a medicine-storing chamber 25, a medicine-administrated mechanism, and a medicine-exiting port. The medicine-administrated mechanism includes a pusher 40, an air intake 45, a medicine-mixing chamber 15; the medicine-exiting port includes a nose-inserting portion 10. However, the differences between the aforesaid embodiment and the present embodiment are that the trough of the pusher of the present embodiment is characterized as a hollow column, the front portion of the pusher 40 has a design of handgrip teeth thread 35b, and the portion of the pusher 40 that is close to the medicine-mixing chamber 15 has a design of screw thread 40b. Wherein, when the pusher 40 is screwed and pushed forward by the handgrip teeth thread, the powder-like medicine 100 will drop into the screw thread gap 20a of the screw thread 40b that is attached to the pusher 40. And, therefore the powder-like medicine 100 follows the proceeding motion of the pusher 40 and drops into the medicine-mixing chamber 15, then is inhaled into the nostril through the medicine-inhaling hole 5, enters into human body and adheres to the mucous membrane of the respiratory tract.

In summarizing above-description, the respiratory tract medicine-administrated apparatus and application method of the present invention are that the respiratory tract medicine-administrated apparatus is inserted into the nostril, and inhaling, the air was compressed through the inhaling hole when entering the medicine-mixing chamber and induce the air whirl to mix powder-like medicine with air, which is inhaled altogether into the nostril through the medicine-inhaling hole, enters into the human body and adheres to the mucous membrane of the respiratory tract. Therefore, the method for the medicine-administrated apparatus in respiratory tract of the present invention applied to the medicine of the human respiratory tract is that the powder-like medicine directly enters into the human body and adheres to the mucous membrane of the respiratory tract to be able to destroy or suppress the bacteria inside the respiratory tract, effectively prevent the bacteria from damaging the human body respiratory organs, further suppress the virus self-reproduction and greatly enhance the potency of the medicine. And, the material of the medicine-administrated apparatus of respiratory tract of the present invention is a transparent resin, which can be provided for clearly observing the administrated situation of the powder-like medicine. Additionally, the respiratory tract medicine-administrated apparatus of the present invention may provide several times of usage continuously, and further facilitate the usage for patients due to easily carrying.

The present invention applies a respiratory tract medicine-administrated apparatus to proceed with the medicine administrating method for the respiratory tract with human body ‘inhaling type’ powder-like medicine that enters into the respiratory tract and adheres onto the mucous membrane. Its effect is much stronger than that of an antibiotics medicine of oral type, which is well known to everybody. Usually, the oral type medicine has apparent effect only after several days of medicine taking and the needed quantity of medicine exceeds hundreds times of quantity of the present invention. The oral type of medicine taking is not only an extreme burden for human body, but also generates the problems of drug-resistance and the waste of medicine cost. The method of the present invention not only has no above-mentioned harassment at all, but also may generate significantly curing effect immediately. Furthermore, the present invention applies the apparatus and method, which human body ‘inhaling type’ powder-like medicine enters into the respiratory tract and adheres onto the mucous, membrane and which are different from the prior apparatus and method of medicine entering human body with ‘spraying type’, and which have much stronger curing effect.

Of course, the respiratory tract medicine-administrated apparatus of the present invention is not restrained to the usage for being inserting into the nostril, but may be operated by inserting the nose-inserting portion of the respiratory tract medicine into the mouth of human body to make the powder-like medicine be successfully inhaled into the respiratory tract. Furthermore, the respiratory tract medicine-administrated apparatus of the present invention may be widely applied to the medicine-administrated methods for any kinds of viral disease of respiratory systems, especially for novel virus of mutation or rare species without antibodies inside human body, and the respiratory tract medicine-administrated apparatus of the present invention may provide the best medicine-administrated method for all these kinds of diseases.

The method of the present invention makes the immune system of human body do not need to fight against the bacteria during the initial phase of influenza infection with all its strength, but makes the virus directly exposed to the first battle line of the immune system. Thus, the macrophage may exhaust all its strength to fight against and phagocytize up the influenza virus and make plasma cells generate the antibody of influenza virus. Additionally, the said response of human complement system not only may dissolve the virus envelope to kill the virus, but also may further utilize the opsonization completely to enhance the effect and the rate for phagocytizing the virus.

Of course, above-mentioned descriptions are only the preferable embodiments for the respiratory tract medicine-administrated method and the respiratory tract medicine-administrated apparatus for continuous usage of the present invention, and which are not used for restraining the implement scope of the present invention. Any kind of change that is made and not contradicted to the spirit of the present invention by any person familiar with these techniques is all belonged to the scope of the invention. Therefore, the protecting scope of the present invention should be based upon following said claims.

What is claimed is:

1. A method of medicine administration for respiratory tract, including:
providing powder-like antibiotics;
making the powder-like antibiotics enter into the front portion of the respiratory tract of human body by a medicine-administrated method; and
making the powder-like antibiotics enter into the interior of respiratory tract of human body and adhere onto the mucous membrane by the inhaling motion of human body.

2. The method of medicine administration for respiratory tract according to the claim 1, wherein the powder-like antibiotics could be replaced by a powder-like bacteriophage.

3. The method of medicine administration for respiratory tract according to the claim 1, wherein the medicine-administrated method is referred to a respiratory tract medicine-administrated apparatus, which sends medicine into the respiratory tract of human body.

4. The method of medicine administration for respiratory tract according to the claim 3, wherein the respiratory tract medicine-administrated apparatus including:
a medicine-storing chamber, which has a bottom portion opening;
a pusher, which is inserted into a pusher trough that is connected with the bottom portion opening of the medicine-storing chamber;
a medicine-mixing chamber, of which one side portion is connected to the trough and another side is a nose-inserting portion; and
an air intake, of which the bottom portion is a contracting opening that, is connected to the medicine-mixing chamber, and another side is an opening.

5. The method of medicine administration for respiratory tract according to the claim 4, wherein the front end of the pusher of the respiratory tract medicine-administrated apparatus is attached with a handgrip.

6. The method of medicine administration for respiratory tract according to the claim 4, wherein a design of spring is attached onto the pusher of the respiratory tract medicine-administrated apparatus.

7. The method of medicine administration for respiratory tract according to the claim 4, wherein the rear portion of the respiratory tract medicine-administrated apparatus has a design of medicine-retrieving portion with opening hollow shape.

8. The method of medicine administration for respiratory tract according to the claim 4, wherein the most front end of the nose-inserting portion of the respiratory tract medicine-administrated apparatus is arranged with a medicine-inhaling hole with opening shape.

9. The method of medicine administration for respiratory tract according to the claim 4, wherein the respiratory tract medicine-administrated apparatus is a material of transparent resin.

10. The method of medicine administration for respiratory tract according to the claim 4, wherein two sides of the pusher of the respiratory tract medicine-administrated apparatus are arranged with pusher grooves, and two side openings of the pusher trough are arranged with tenons, which could be inserted into the pusher grooves.

11. The method of medicine administration for respiratory tract according to the claim 4, wherein the pusher trough of the respiratory tract medicine-administrated apparatus could be a hollow column.

12. The method of medicine administration for respiratory tract according to the claim 11, wherein the front portion of the pusher of the respiratory tract medicine-administrated apparatus has a design of handgrip teeth thread, and the portion of the pusher close to the medicine-mixing chamber has a design of screw thread.

13. A apparatus of medicine administration for respiratory tract applied to the claim 1, including:
a medicine-storing chamber, which is attached with an opening for filling in medicine;
a medicine-administrated mechanism, which is connected to the medicine-storing chamber; and
a medicine-exiting port, which is connected to the medicine-administrated mechanism.

14. The apparatus of medicine administration for respiratory tract according to the claim 13, wherein the medicine-administrated mechanism including:
a pusher, which is inserted into a pusher trough that is connected with the bottom portion of the medicine-storing chamber;
a medicine-mixing chamber, of which one side portion is connected to the trough and another side is connected to the medicine-exiting port; and
an air intake, of which the bottom portion is a contracting opening that is connected to the medicine-mixing chamber, and another side is an opening.

15. The apparatus of medicine administration for respiratory tract according to the claim 14, wherein a most front end of the air intake is arranged with a nose-inserting portion with opening-shaped medicine-inhaling hole, and the nose-inserting portion is inserted into the nose or mouth and inhale air.

16. The apparatus of medicine administration for respiratory tract according to the claim 14, wherein the front end of the pusher is attached with a handgrip.

17. The apparatus of medicine administration for respiratory tract according to the claim 14, wherein a design of spring is attached onto the pusher.

18. The apparatus of medicine administration for respiratory tract according to the claim 14, wherein two sides of the pusher are arranged with pusher grooves, and two side openings of the pusher trough are arranged with tenons, which is inserted into the pusher grooves.

19. The apparatus of medicine administration for respiratory tract according to the claim 14, wherein the pusher trough is a hollow column.

20. The apparatus of medicine administration for respiratory tract according to the claim 19, wherein the front portion of the pusher has a design of handgrip teeth thread, and the portion of the pusher close to the medicine-mixing chamber has a design of screw thread.
